SEQUENCE LISTING (4 sequences)

SEQ ID NO: 1 - DNA, 5757 bp, Human
  CDS (228)..(3893)
atcaccctctggacaagagaacgggcgagcgggagctaggagggaagagtggagaggaccggcgaggcgcgccagccggagccacctccttcccggccgccccctccccactccccctacacacacacgctcgctcgctcgccggcgcgcgcacaccccccgcgccggacccgcacctcggcgggcgccacacactcggcagcccgagccgcggtagccgcagcgggatggaggcggcgcgcacggagcgccccgcaggcaggccgggggcgccgcttgtccggacggggctcctactcttgtcgacgtgggtcctggccggcgccgagatcacttgggacgcgacaggcggtcccggacgcccggcggccccggcttcgcggccaccggcgttgtctccactctcgccgcgggcagtggccagccagtggccggaggagctggcgtcggcgcggagagccgccgtgctggggcgccgggccggaccagagctgctgccccagcagggcggcggcagaggcggtgagatgcaggtggaagccggagggacatcaccggcaggcgagcggcggggccggggcatcccagctcctgccaagcttggcggcgcgaggaggagtcgccgggcgcagcccccaatcacccaggaacgcggggacgcctgggccactgctccggccgatggttccagaggaagccgtccccttgctaagggttcccgggaggaggtgaaggcgccgcgggctggggggtcggcggctgaagacctccggctgcccagcacctccttcgcgctgaccggggactcggcccacaaccaagccatggtgcactggtcgggacacaacagcagcgtcatacttatcctgacgaagctgtatgacttcaacctgggcagcgtgactgagagttcactatggaggtcgacagattatggcaccacctatgaaaagctgaatgacaaagtgggtttgaagactgtcctcagttacctctatgtcaatccaaccaacaaaaggaagattatgcttctcagtgatcctgagatggagagcagcatattgatcagctcagacgaaggggcgacctatcagaagtatcggctcaccttctatatccagagcctgctctttcatcccaagcaagaggactgggtgctggcctacagtttggatcaaaagctctacagctccatggactttggaagacggtggcaactcatgcatgaacgcatcacacccaacaggttttattggtcggtggccggattggataaggaggcggacctggtgcacatggaggtgcggaccacggatggatatgctcactacctcacctgcaggatccaggaatgtgccgagacaactagaagtgggccttttgcccgctccattgacatcagttccctggttgtccaggatgaatatatcttcattcaggtaacaactagtggaagagccagctactacgtgtcttatcgaagagaggcctttgctcagataaagctgcctaagtactcgttgccaaaggacatgcacatcatcagtacagacgagaaccaagtatttgctgcggtccaagaatggaaccagaatgacacgtacaacctctacatctcagacacgcgtgggatttacttcactctggccatggagaacatcaagagcagcagaggtctaatggggaacatcattattgaattgtatgaggtagcaggtatcaaagggatatttctggcaaacaagaaggtggacgaccaggtgaagacatacatcacttacaacaaaggcagggattggcgcctgctgcaagctccggatgtggacctgagaggaagcccagtgcactgcctgctgcccttctgttccttacatctgcacctgcaactctctgaaaatccatattcctcaggaagaatctctagcaaggagacagccccaggacttgtggtggctacaggcaacattggcccggagctctcatatactgatattggtgtgttcatctcctccgatgggggcaacacatggagacagatctttgatgaagagtacaatgtctggttcctagactggggtggtgccctcgtggccatgaaacacacacctctgccagtcaggcatttgtgggtgagttttgatgagggccactcttgggacaagtatggtttcacttcggttcctctctttgttgacggggctctggtggaggcaggaatggagacccacatcatgacagtttttggccacttcagcctccgctccgaatggcaattggtgaaagtggactacaaatctatcttcagccggcattgcaccaaggaggactatcagacctggcacctgctcaatcagggagagccttgtgtcatgggagaaaggaaaatattcaagaaacgtaagccaggagctcagtgtgccctgggccgagaccactcaggatcagtggtctcagaaccctgtgtctgtgccaattgggacttcgagtgtgactatgggtatgagagacatggggagagccagtgtgtcccagctttctggtacaatccagcatccccatcaaaggactgcagccttggtcaaagctaccttaacagcactgggtatcggcggattgtgtccaacaactgcacagatgggctaagggagaagtacaccgccaaggcccagatgtgccctggaaaagcccctcggggcctccatgtggtgacgaccgatgggcggctggtggcagagcaggggcacaatgcaactttcatcatcctcatggaggagggtgatctacaaaggacaaacatccagcttgactttggggatgggattgctgtgtcctacgcaaacttcagccccatcgaggacggcatcaagcacgtgtataagagtgcggggatcttccaggtgacagcctatgcagagaacaaccttggctcagacacagctgtcctcttcctgcatgtggtttgtcctgtggagcatgttcatctccgagttccatttgttgccataagaaataaggaggtcaacatcagtgcagtcgtgtggcccagtcaactggggacccttacctatttctggtggttcggcaatagcacaaagcctctcatcactttggacagcagcatttccttcacattccttgcagaaggaaccgacaccatcacagtccaggtggctgctgggaatgccctcatccaggacacaaaagagattgcagttcatgaatatttccagtcccagcttttatcattctctcctaatctggattaccacaatcctgacattcctgagtggagaaaagatattggcaatgtcatcaagcgagctctggttaaagtaaccagtgtcccagaggaccagatcctcattgccgtgtttcctggtctccccacttcagcagagcttttcattcttccacccaagaacctgacagagaggaggaaaggcaatgaaggggacctggaacaaattgtagaaacactgtttaatgctctcaaccaaaatttggtccagtttgagctgaagccgggggtacaagtcattgtgtatgtcacacagctgacgttagctccattggtggactccagtgctgggcacagcagctcagccatgcttatgctattatcagtggtatttgttggcctggctgtgtttttgatctacaagtttaaaaggaaaatcccttggattaacatctatgctcaagtccaacacgacaaggagcaggagatgattgggtcagtgagccaaagtgaaaacgcccccaaaatcacactcagtgactttacggagcctgaggagctgctggacaaagagctggacacgcgggtcataggaggcattgccactattgcaaacagcgaaagcacaaaggagatccccaactgcactagtgtttaataccagcaagccacgtggtcaaccacctttctgactttttatttttgatgattactattactattattatggaaaaattaaaatgtcttttttaccttttgtttaccaagggccccttcataaatagcaggcaaatgcctagctttgggagaaaagggcattcttagctgattgaaatgagacaaagggaataaatggctgtatttgtgctaagagcaaaggatgcatcttcccacagcctcctcgctttactctgccattggtagcttaaagactttctttttccttgtggtctcccttttttcaaaattgaagttgggttggctctttgtgaacctctcatccccacagcagaatcaccaacactctccgcttcccccagcacacacacatacaacacagatcatttcccagttagatccgcaggaagtaggttggtgggggtggatgtagctgcagaaagcatgcacaactttgtgaaagaggccctgccttgtgcatgtccatagtgaggctacagatggcttattgtatataattacaatgtaaatagctttttatttcctaagaaataatttaatgtttagtaaaaaagaaaacagaaaaaagaaagatgcgtgtgttggcttacgcactggccctcagagctgaccaacccgccaggcctgctcaatgcattgggtttggatgctctcctgttgtctgtcacacttaactcttgcatctccttgtccatgccatagctggtttctacttatgtatataaaggggggtggggggaggggcttctctggggcaattgataaaggaaggactctagtgacatcatagaacatggcagtcgtttttgttccaagaatgatatgaaaggtgaagaagaggcccactagaggcttcatactgagacccagatgggggaaaacagcttcctctctaaaaggaaaaacttgatatttatcagtctgagaaaatatttttttctaaagaaggcagtcagtggatcttaaaatgacaatctgtttttaaattggattctatgaaaatgcataatgcttatggtgaattctcaggctattctgagctcagaaaagtcccctgggcactaggtaaagcccagtgaatgtctcttggcatgggaggagttaaagaggttggaagggaagaggcatttgtggaattatgagttcatgcaaaactctccaggccaagtaggggtctagcctttaatgatattagtcaaaggcaattttagcaaagctgtgctatttgcttgtcagatgtacacaacttccttaaagtcaaatgtctgccttcagttcccttaaggtagttcttgcctctggggtgagtggctttcaaagccttttagcttttccagcacctcagccccttcacacatttacacataccaatttttttcaatagggtcacgttaagccatgctgtaagcattgtttttattttcaggcttagcctgagcacacttatttttgaaaatgatataatgtatatatatgggaggaaaggccacattttgtacctgttaatttttgtgggatgttgttcccattcttctttgtgagacagagagaatgtgatatagagaaatctggctggctacagtgtagatcagtattaggaatatttctaaagatcctgcttttttgtttcaagggttaaatggggcagacaattgcaatacttgtactaaacactggaatacaaatgcatgactcatatctatatatacagtatatgtacatatactgttcttggttttattgttccacttgaatatttctactgtaaaaaaaagacagtggttttgaaattgttgaaaataaatgtatttttgtacatc

SEQ ID NO: 2 - protein, 1222 aa, Human
MEAARTERPAGRPGAPLVRTGLLLLSTWVLAGAEITWDATGGPGRPAAPASRPPALSPLSPRAVASQWPEELASARRAAVLGRRAGPELLPQQGGGRGGEMQVEAGGTSPAGERRGRGIPAPAKLGGARRSRRAQPPITQERGDAWATAPADGSRGSRPLAKGSREEVKAPRAGGSAAEDLRLPSTSFALTGDSAHNQAMVHWSGHNSSVILILTKLYDFNLGSVTESSLWRSTDYGTTYEKLNDKVGLKTVLSYLYVNPTNKRKIMLLSDPEMESSILISSDEGATYQKYRLTFYIQSLLFHPKQEDWVLAYSLDQKLYSSMDFGRRWQLMHERITPNRFYWSVAGLDKEADLVHMEVRTTDGYAHYLTCRIQECAETTRSGPFARSIDISSLVVQDEYIFIQVTTSGRASYYVSYRREAFAQIKLPKYSLPKDMHIISTDENQVFAAVQEWNQNDTYNLYISDTRGIYFTLAMENIKSSRGLMGNIIIELYEVAGIKGIFLANKKVDDQVKTYITYNKGRDWRLLQAPDVDLRGSPVHCLLPFCSLHLHLQLSENPYSSGRISSKETAPGLVVATGNIGPELSYTDIGVFISSDGGNTWRQIFDEEYNVWFLDWGGALVAMKHTPLPVRHLWVSFDEGHSWDKYGFTSVPLFVDGALVEAGMETHIMTVFGHFSLRSEWQLVKVDYKSIFSRHCTKEDYQTWHLLNQGEPCVMGERKIFKKRKPGAQCALGRDHSGSVVSEPCVCANWDFECDYGYERHGESQCVPAFWYNPASPSKDCSLGQSYLNSTGYRRIVSNNCTDGLREKYTAKAQMCPGKAPRGLHVVTTDGRLVAEQGHNATFIILMEEGDLQRTNIQLDFGDGIAVSYANFSPIEDGIKHVYKSAGIFQVTAYAENNLGSDTAVLFLHVVCPVEHVHLRVPFVAIRNKEVNISAVVWPSQLGTLTYFWWFGNSTKPLITLDSSISFTFLAEGTDTITVQVAAGNALIQDTKEIAVHEYFQSQLLSFSPNLDYHNPDIPEWRKDIGNVIKRALVKVTSVPEDQILIAVFPGLPTSAELFILPPKNLTERRKGNEGDLEQIVETLFNALNQNLVQFELKPGVQVIVYVTQLTLAPLVDSSAGHSSSAMLMLLSVVFVGLAVFLIYKFKRKIPWINIYAQVQHDKEQEMIGSVSQSENAPKITLSDFTEPEELLDKELDTRVIGGIATIANSESTKEIPNCTSV

SEQ ID NO: 3 - DNA, 7272 bp, Human
  CDS (9)..(3512)
ctcccgcgatgggaaaagttggcgccggcggcggctcccaagcccggctgagcgcgctcctcgccggcgcggggctcttgatcctctgcgccccgggcgtctgcggcggcggctcctgctgcccctcgccgcaccccagctccgctccacgctcggcctcgacccctaggggcttttcccaccaggggcggccaggcagggctcctgccacgcccctgcccctcgtagtgcgtcccctgttctcagtggcccccggggaccgagcgctatccctggagcgggctcggggcactggggcatccatggcggttgctgcacgctccggccggaggagacggagcggagcggatcaggagaaggcagaacggggagagggcgcgagtcggagcccccggggagtgctaagagatggagggcagcaggagcctgggactcgggagcgggacccggacaaagccacccgcttccggatggaggagctgagactgaccagcaccacgtttgcgctgacgggagactcagcacacaaccaagccatggtccactggtctggccacaacagcagcgtgattctcattttgacaaagctctatgactataacctggggagcatcacagagagctcgctttggaggtcaaccgattatggaacaacctatgagaagctgaatgataaagttggtttgaagaccattttgggctatctctatgtgtgtcctaccaacaagcgtaagataatgttactcacagacccggagattgagagcagtttattgatcagctcagatgaaggggcaacttatcaaaagtaccggctgaacttctacattcaaagcttgctttttcaccccaaacaagaagactggattctggcatacagtcaagaccaaaagttatacagctctgctgaatttgggagaagatggcagcttatccaagaaggggttgtaccaaacaggttctactggtctgtgatggggtcaaataaagaaccagaccttgtgcatcttgaggccagaactgtggatggtcattcacattatctaacttgccgaatgcagaactgtacagaggccaacaggaatcagccttttccaggctacattgacccagactctttgattgttcaggatcattatgtgtttgttcagctgacatcaggagggcggccacattactacgtgtcctaccgaaggaatgcatttgcccaaatgaagcttccgaaatatgctttgcccaaggacatgcatgttatcagcaccgatgagaatcaggtgttcgcagcggtccaagaatggaaccagaatgacacgtacaacctctacatctcagacacacgtggtgtctacttcaccctggccttggagaatgtccagagcagcagaggccctgagggcaacatcatgatcgacctctatgaggtagcagggataaagggaatgttcttggctaacaagaagattgacaaccaagtgaagactttcatcacatataacaaaggcagagactggcgtttgctgcaggcgccggacacggatctaaggggggaccccgtgcactgcttgctgccctattgctcactacaccttcacctgaaggtctctgagaatccctacacatcagggatcattgccagcaaagacacagctccaagcatcatagtggcatcaggtaatataggttctgaattgtcagacactgacatcagcatgtttgtctcttcagatgcagggaacacctggagacagatctttgaagaagagcacagtgttttgtacctggatcaaggtggagtcctggttgctatgaaacacacatctctcccaattcgacatctttggttgagttttgatgaagggagatcttggagcaaatacagtttcacatctattccactttttgtggatggggttctgggtgagcctggagaagagactctcatcatgacagtgtttggacacttcagccaccgctctgaatggcagctggtcaaagtagattacaagtccatttttgatagacggtgtgccgaagaggactacagaccttggcagctgcacagccagggggaagcatgtatcatgggagcaaaaaggatatataagaagcgaaaatcagagcggaagtgtatgcaaggaaaatatgcaggagctatggaatctgaaccctgtgtctgcactgaggctgattttgattgcgactatggttatgagcgacacagcaatggccagtgcctgccggcattttggttcaatccatcctctctgtcaaaggattgcagcttgggacagagttacctcaatagtactgggtacaggaaggtggtttccaataattgcactgatggcgtaagggaacagtacactgccaaaccgcagaagtgcccagggaaagccccgcgggggctgcggatagtcacggctgatggaaagctgacagcggaacaaggacacaacgtcactctcatggtgcaattagaagagggtgatgttcagcggacactcatccaagtggactttggcgatggtatcgcggtgtcttacgtcaatctcagctccatggaagatgggatcaaacacgtctatcagaacgtgggcattttccgtgtgaccgtgcaggtggacaacagtctgggttctgacagcgccgtcctgtacttacatgtaacttgtcccttggagcacgtgcacctgtctcttccctttgtcaccacaaagaacaaagaggtcaatgcgacggcagtgctgtggcccagccaagtgggcaccctcacttacgtgtggtggtacggaaacaacacggagcctttgatcaccttggagggaagcatatccttcagatttacttcagaaggaatgaataccatcacagtgcaggtctcagctgggaatgccatcctacaagacacaaagaccatcgcagtatatgaggaattccggtctcttcgcttgtccttttctccaaacctggatgactacaacccggacatccctgagtggaggagggacatcggtcgagtcatcaaaaaatccctggtggaagccacaggggttccaggccagcacatcctggtggcggtgctccctggcttacccaccactgctgaactctttgtcctaccctatcaggatccagctggagaaaacaaaaggtcaactgatgacctggagcagatatcagaattgctgatccacacgctcaaccaaaactcagtacacttcgagctgaagccaggagtccgagtccttgtccatgctgctcacttaacagcggcccccctggtggacctcactccaacccacagtggatctgccatgctgatgctgctctcagtggtgtttgtggggctggcagtgttcgtcatctacaagtttaaaaggagagtagctttaccctcccctccctccccttctactcaacctggtgactcatctctccgattgcaaagagcaagacacgccactccgccttcaacgccaaagcggggatctgctggggcacagtatgcaatttaaggaaaacccccaaaggctacaggcgacctgctgatcaggaaagaatttcgctcttgtcaagtacatcatccttcatgaccactaactttgtgttttttttctttcctttgttgttctgtttcctattttgccaggaagtatttccatagttgctgagaatcaaagcacaaaagaaatccctacctatgtaaatgtttgaatggaggacgccagtaaaaaaacaaaaacaaaaacaaaacaaaacataaaatataaacaatcaaaatccaaacaaacaaacaaacactcactgcatcgggactttttaattcttcagacacagacaacaagggtttttagctttaagcctgtgcatgtggacaatactctgagaacatgtctggaggggcagtgtacaggtgctctattttaatggaaaacactcccctctccctctttcttcttctctctcttttttctgatcggtcgtgtttgtagaaaattcataacatatataggccaaggaaatctgcatgtatttttggaaatattcttggctctagatttatcagctattttagcattaaaggctgatgggtggattagctaccccagctcctttcataagacacaaagacgtgcacaggagtttgaaaccctgagctgtttctctgttccactttccatactgtcatttcccttctaagttagctttggtagctctgttggtcagcagtggccacaaggctgctttctgcactctctgtggccacaggaacaaagatgcgagttgaagatccctttgtgccggtagaataaggaaggaagggaggcagggaggcaaagcatgtcagaaacggaccttggtttcctgttcacctctttccaccatgattgcctccccttgcagctttccctactcctgccccaactgcagtaggaaatggaatcccattagttacccattgtcctgtcttcacatttgtttgtcatccatgtgtgaccatgatctgttgatatctttgaatctcttccctccaaaccccaagtaagttgtccttcaactgttctcagtttttcttcccaaacatattgctagattctggacattaaccctgacgtttcctaaatactggcctggcctgtgccgcccctgcccccactactcacaagtttctgagccctttgtcagttttgtacattcccaagcatgccagcttctgtgcccatggaccttgctgcatcctgcacagcagggttcagtttcatgtatctttgtcttttcctctagaacctgcctttttcaaatatccctgccttttcccacctggaaacatatactgtatgcgaaagagttgatatatgcaaagtaattcaaacctggccatgtactttggaaaagaaagcatagagatgaattgtggtgttctcacaccttaacgaaaatctcgtgcgatctgattccagaagattcttgtgagaaatgttttgaatgtgtgacaatttcgcagggcaatttcctcctgtgacggttgctgttacctggtatttccactctcacagtagaatagaaatgtttgtgaaataaaactgattttaactggaaggagaaacaggttaatggactttgtgtttaagagtgtcaaacagtctgagagaataaatgggggtcttgtcttacattagggtgagagtttgattatttaggatgatcccagtgatttcatgtgtgctctctgacctctgagtatcatagtatcattaaagtaaagatttaagtctgtataaaggaggagagttactgattggcaaaaaagattggcacaaagcatgaagaaaccccatttttcccagggtaatcatgaaagaaggctcagagaaagagggaaacaaaagcctgttcagcagaggcccctttagtattatggactgggcaaagcccactataaactataggagaaagaagttctgataaacctcttagtatgctcagcctccttctttgctatgtccctaagccaacagggtataacacaggccctggtgataatgagggtgtcctaaagatcctgatgtccatgacttcatgtagttcaggcacagaaagaatagcaaagctgctgtcagaagtttgtcaggcagagatgtttatggttgaggataccccatatgaatctgggaaatgggctaggcctgaggcagtcacctatttgtagaactcaattgatccccaacctgccaggcttcctatctcataggaaacatcctcattgatcctcttcagttggagtctcccaaatttaatgtggaagacaaagtgttggaaaatcaaaagaggctcactcaaaccagcacagggagtcctcagagttgcagttcaattcattttaattagaaaagtatgaaagaggtataacactcttattctagcacagtgccttgcctagagtagtagtatgaaaactatttgttgaagaaataaatgaatggaaagacatagggaagaatgggctgtaaatcctaattaaaaatagaggtcgacatgagatacctggcattttgggaagtgaccaaagatggccagctagagattcagcatctccaggatcctcatgtgcctctcctcaaagctccctcccatctgtaggagattagtgaggcaaggtgctgctcagagaggaggacctcattgttcttaggaccttggccagttgttctcaaagtgtggtacctggatcagcagtatcactatcacttgggctagttagagatccagattctggatccataccccatacctactgaattagaaactctaggggatgaatagtggtgttctctgttgctaagtgacagtgggcccagccacctattttaacaagccctcccagtgattgtgatacatactaaagtttgagaaccattgtactaggccattccagctgaatctcaaacagaaggcagtaatgagagcctacaaatgggagggacctaagtgcctacctactcgctaatcgcaggtgcaaacacacaaggagtttggtgggcttaaggtcagaggagtgtgtagggagggatgtatgtggaaggtaagattcagggcaagctaaaaatccgatactgcaacgttttccaaaatcccagaaggcaaactgtgcatgttctaccctgaaccacccaagcaacactttctaccttgccttatttttaattggattcactgtccaaaatgcagaggtttgctttgcttttttttcagaagttccaaacagcaactttgagagcagtggggtgcttggcagctgttctgtgttttccaggaatccaactgagcattgaaatctctcatttgccgacttatttttataggaagccaattaaaaaaaaaaaaagttttcttatagtattggaactacttctaattttaaaatgacttttttgatgtattttttgttaaatactatgtagtgtaatgtataattgctcttgtttattgcttttacaatcatatttattaaacagataatgtctctaaagtctttgcctcaggtatttttttttttaatcctaaacccttggtgttcattctaaatatagaagtgttgcatgtataggatttcataaaggctaattgcataagaaagagtaaacaccacaggcttgaggtttttggctgttttttactaacaaggcagaatgtatgtactacctgaattctacctgcatttcaattaactatacaatgtctgtttattaaattactttgatttaaaaatta

SEQ ID NO: 4 - protein, 1168 aa, Human
MGKVGAGGGSQARLSALLAGAGLLILCAPGVCGGGSCCPSPHPSSAPRSASTPRGFSHQGRPGRAPATPLPLVVRPLFSVAPGDRALSLERARGTGASMAVAARSGRRRRSGADQEKAERGEGASRSPRGVLRDGGQQEPGTRERDPDKATRFRMEELRLTSTTFALTGDSAHNQAMVHWSGHNSSVILILTKLYDYNLGSITESSLWRSTDYGTTYEKLNDKVGLKTILGYLYVCPTNKRKIMLLTDPEIESSLLISSDEGATYQKYRLNFYIQSLLFHPKQEDWILAYSQDQKLYSSAEFGRRWQLIQEGVVPNRFYWSVMGSNKEPDLVHLEARTVDGHSHYLTCRMQNCTEANRNQPFPGYIDPDSLIVQDHYVFVQLTSGGRPHYYVSYRRNAFAQMKLPKYALPKDMHVISTDENQVFAAVQEWNQNDTYNLYISDTRGVYFTLALENVQSSRGPEGNIMIDLYEVAGIKGMFLANKKIDNQVKTFITYNKGRDWRLLQAPDTDLRGDPVHCLLPYCSLHLHLKVSENPYTSGIIASKDTAPSIIVASGNIGSELSDTDISMFVSSDAGNTWRQIFEEEHSVLYLDQGGVLVAMKHTSLPIRHLWLSFDEGRSWSKYSFTSIPLFVDGVLGEPGEETLIMTVFGHFSHRSEWQLVKVDYKSIFDRRCAEEDYRPWQLHSQGEACIMGAKRIYKKRKSERKCMQGKYAGAMESEPCVCTEADFDCDYGYERHSNGQCLPAFWFNPSSLSKDCSLGQSYLNSTGYRKVVSNNCTDGVREQYTAKPQKCPGKAPRGLRIVTADGKLTAEQGHNVTLMVQLEEGDVQRTLIQVDFGDGIAVSYVNLSSMEDGIKHVYQNVGIFRVTVQVDNSLGSDSAVLYLHVTCPLEHVHLSLPFVTTKNKEVNATAVLWPSQVGTLTYVWWYGNNTEPLITLEGSISFRFTSEGMNTITVQVSAGNAILQDTKTIAVYEEFRSLRLSFSPNLDDYNPDIPEWRRDIGRVIKKSLVEATGVPGQHILVAVLPGLPTTAELFVLPYQDPAGENKRSTDDLEQISELLIHTLNQNSVHFELKPGVRVLVHAAHLTAAPLVDLTPTHSGSAMLMLLSVVFVGLAVFVIYKFKRRVALPSPPSPSTQPGDSSLRLQRARHATPPSTPKRGSAGAQYAI

(19) United States
(12) Patent Application Publication
(10) Pub. No.: US 2004/0115702 A1
(43) Pub. Date: Jun. 17, 2004

(54) Type 2 diabetes susceptibility genes

(76) Inventors: Alan D. Attie, Madison, WI (US); Jonathan P. Stoehr, Madison, WI (US); Kathryn L. Schueler, Madison, WI (US); Susanne Michelle Clee, Madison, WI (US)

Correspondence Address: Nicholas J Seay, Quarles & Brady LLP, P O Box 2113, Madison, WI, 53701-2113, US

(21) Appl. No.: 10/655,915

(22) Filed: Sep. 5, 2003

Related U.S. Application Data
(60) Provisional application No. 60/409,525, filed on Sep. 9, 2002.

(51) Int. Cl. C12Q 1/68; G01N 33/53

(52) U.S. Cl. 435/6; 435/7.1

(57) ABSTRACT

Two of the genetic bases for susceptibility to type 2 diabetes are disclosed. The alleles of the genes SorCS1 and SorCS3 that a person carries can determine whether or not that person is susceptible to type 2 diabetes.

### CROSS-REFERENCE TO RELATED APPLICATION

This application claims priority from U.S. provisional patent application Serial No. 60/409,525 filed Sep. 9, 2002.

### STATEMENT REGARDING FEDERALLY SPONSORED RESEARCH OR DEVELOPMENT

To be determined.

### BACKGROUND OF THE INVENTION

Type 2 diabetes is also called non-insulin dependent diabetes mellitus (NIDDM) or adult onset diabetes. Over 90% of diabetes is of the type 2 kind. The American Diabetes Association reports that there are 12 million Americans with type 2 diabetes and another 7 million potential candidates. An annual expenditure of $100 billion is attributed to the disease. It is the third leading cause of death at 62,000 each year. Prolonged untreated diabetes leads to heart diseases, stroke, kidney disease, blindness, and loss of limbs from advanced peripheral vascular disease.

Type 2 diabetes involves insulin resistance coupled with failure of the pancreatic β-cells to secret enough insulin to maintain euglycemia (1-3). Although insulin resistance is a feature of type 2 diabetes, an individual can be severely insulin resistant without ever exhibiting fasting hyperglycemia; β-cell insufficiency is an essential feature of type 2 diabetes (4). The question becomes why do some people become severely insulin dependent without developing type 2 diabetes while other people do develop the disease. A logical question becomes whether or not a genetic predisposition to the disease exists.

Obesity is an important independent risk factor for the development of type 2 diabetes: more than 80% of type 2 diabetic patients are obese. Nevertheless, although most obese people are insulin resistant, the majority remains euglycemic. Currently, there are few tools available to help predict which obese individual will progress to type 2 diabetes. Again the question is why some individual are obese and insulin resistant, but not diabetic, while others develop the disease.

Type 2 diabetes does tend to run within families and ethnic groups suggesting a strong genetic contribution to the disease (5). However, the major type 2 diabetes susceptibility genes were heretofore unknown. Identification of susceptibility genes for type 2 diabetes will provide screening tools for identifying individuals who are susceptible to the disease and related diseases so that they can take prophylactic measures. In addition, it can also lead to the development of new prevention and treatment tools for the disease. These tools are used to identify therapeutic agents for the treatment of the disease and related diseases.

### SUMMARY OF THE INVENTION

The present invention is summarized in that a method of assessing whether a human subject is susceptible to type 2 diabetes is based on the step of determining the allele in the genome of that subject of the SorCS 1 gene or the SorCS3 gene.

It is a feature of the present invention that one of the genetic bases for susceptibility to type 2 diabetes has been identified.

It is an object of the present invention to enable genetic tests to determine if individuals have a genetic susceptibility to type 2 diabetes arising from the allele of the SorCS 1 gene or the SorCS3 gene carried by that individual.

Other objects advantages and features of the present invention will become apparent from the following specification.

### BRIEF DESCRIPTION OF THE DRAWING FIGURES

FIG. 1 is a genetic map of a region on mouse chromosome 19 in which the genetic element responsible for susceptibility to type 2 diabetes was found.

FIG. 2 is a best fit genetic comparison of the amino acid sequences of human and mouse SorCS 1 proteins.

### DESCRIPTION OF THE INVENTION

It is taught here that a mammalian gene known as SorCS 1 genes is one of the genetic elements which can make a person susceptible to type 2 diabetes. An alteration to the human SorCS1 gene makes an individual susceptible to developing type 2 diabetes. The mutant form of the gene does not cause type 2 diabetes, there must still be the conditions that lead to insulin insensitivity, such as obesity. The identification of this gene as a contributor to susceptibility to type 2 diabetes begins to answer the questions about why some people develop type 2 diabetes while others do not.

A similar indication has been found about a related gene known as SorCS3. Alterations in the gene and resultant protein for the SorCS3 locus are also indicators of susceptibility for type 2 diabetes in humans.

The identification of thSorCS1 gene as a type 2 diabetes susceptibility gene was worked out in two congenic mice strains, which have a SorCS1 gene directly analogous to the human gene. In summary, two groups of obese mice were identified, a first group which was would develop a severe from of type 2 diabetes and a second group which proved to develop a less severe form of type 2 diabetes. By breeding and genetic testing, the source of the genetic difference between the two groups of mice was identified. Two loci were mapped that determined diabetes susceptibility. One loci was on chromosome 16, where the diabetes-associated allele comes from a diabetes-susceptible mouse strain BTBR, which would develop only the less severe form of diabetes. The second locus was found to be located on chromosome 19, and this allele, carried in a mouse strain B6, was associated with the more severe form of type 2 diabetes. The phenomenon by which a disease trait is transmitted from the unaffected parent to its offspring is termed “transgression.” The strongest data comes from congenic mice where BTBR obese mice are diabetic and the severity of their diabetes in much greater if they inherit a 7 Mb segment of chromosome 19 from a B6 parent. The mice exhibited very high levels of plasma glucose, averaging 120 mg/dl, more than the glucose level of BTBR obese mice. It was ultimately determined that the more severely diabetic mice have an allele of the SorCS 1 protein (the B6 allele) that is three amino acids different from the allele of that same protein in the BTBR mice which would not develop type 2 diabetes to the same degree of severity. In other words, the difference in susceptibility to severe diabetes resolved down to differences in the allele of the gene for SorCs1. Since the same phenomenon exists in humans, and the analogous SorCS1 gene is found in humans, the same variation in severity of diabetes is found in humans. Thus it is now possible to perform genetic tests of human individuals and determine if the patient is genetically susceptible to severe type 2 diabetes due to his or her allele of the SorCS1 gene. Note that this gene is one of the sources of genetic susceptibility to type 2 diabetes, but it may or may not be the source of all such susceptibility. It is possible that there are other genes which contribute to the genetic susceptibility to this disease. What can be said here is that this gene is at least one of the sources of genetic susceptibility to type 2 diabetes, and that allelic differences in this gene are alone sufficient so explain some of the genetic susceptibility to the disease.

The identification of this two gene as a trait for susceptibility to severe type 2 diabetes suggests new diagnostic, prevention and treatment tools for type 2 diabetes and related diseases. Related diseases include those diseases and conditions which are treated or ameliorated by modulation of SorCS1 activity or expression. These diseases and conditions include type 1 diabetes, and other disorders relating to glucose metabolism, insulin secretion, insulin degradation, vesicle transport in secretory cells, pancreas and hepatocyte activity, dyslipidemia and obesity.

As described in the example below, inventors began by narrowing down the genetic region associated with a genetic cause of severe type 2 diabetes to a 7 Mb segment of mouse chromosome 19, to allow the identification of genes that are associated with the severe form of type 2 diabetes. Two genes previously were found in that region, SorCS1 and SorCS3. SorCS 1 and SorCS 3 belong to the sortilin gene family which include sortilin, SorCS1, SorCS2, SorCS 3 and sorLA. The genes in this family share a large region of similarity including the VPS10 domain. Sortilin is located in vesicles in muscles and adipose tissue that contain glut4. Glut4 is the insulin sensitive glucose transporter that is shuttled to the cell surface upon insulin stimulation to enable cells to import glucose at a higher rate. Sortilin also binds to lipoprotein lipase and a neuropeptide called neurotensin through the VPS10 domain. Thus, SorCS3 and SorCS1 are expected to be involved in insulin-stimulated glucose transportation and in controlling body fat metabolism. To verify which of these genetic elements was responsible for the difference in susceptibility to diabetes required characterization of the genes and the alleles present in those gene.

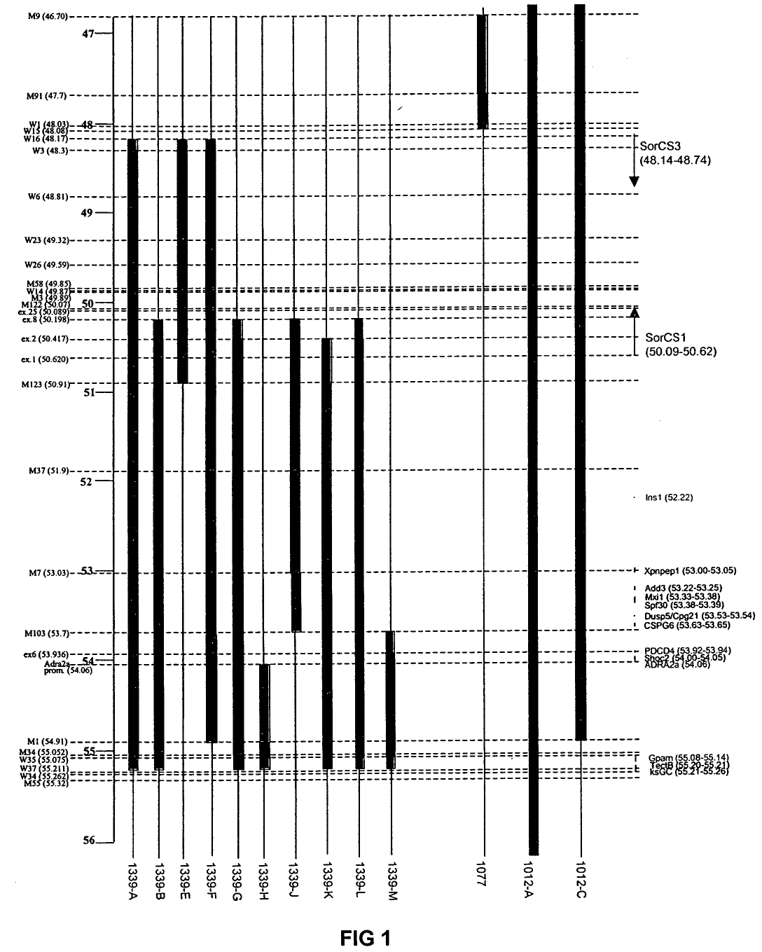

The inventors thus proceeded to characterize the genes and sequences in the 7 Mb region. It was discovered that for each of the genes present, the alleles of the genes carried by the most severely diabetic mice was the same as the alleles of the genes carried by the less severely affected mice, with the sole exception of the allele of the SorCS1 gene. FIG. 1 illustrates a genetic map of the genetic elements found in the 7 Mb region associated with the genetic difference. The region between map units 55 and 48 carried the genetic difference. The alleles of the SorCS3 gene turned out to be identical in the two strains of mice. As illustrated in Table 1 below, however, the susceptible mice had an allele of the SorCS1 gene that is three nucleotides different from that of the less severely diabetic mice. The resulting protein is also three amino acids different. This difference results in a genetic susceptibility to type 2 diabetes.

TABLE 1
SorCS1 mutations altering amino acids

| Nucleotide | | | Amino Acid | | | |
|---|---|---|---|---|---|---|
| position | | | position | | | |
| in cDNA | B6 | BTBR | in protein | B6 | BTBR | isoform(s) |
| 172 | C | T | 50 | Thr | Ile | a, b, c |
| 3433 | C | T | 1139 | Ser | Phe | a |
| 3462 | T | C | 1149 | Ser | Pro | c |

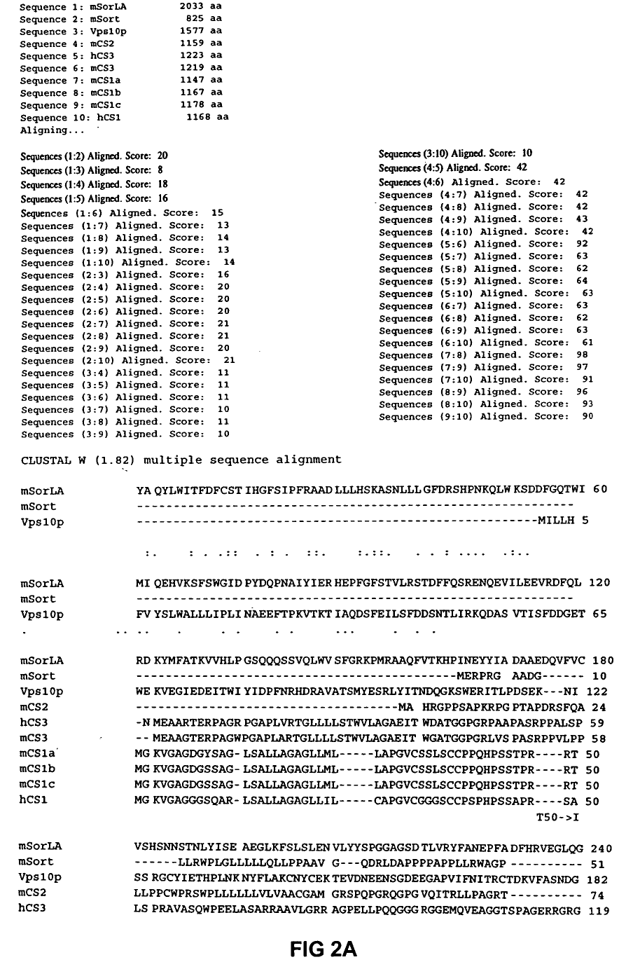

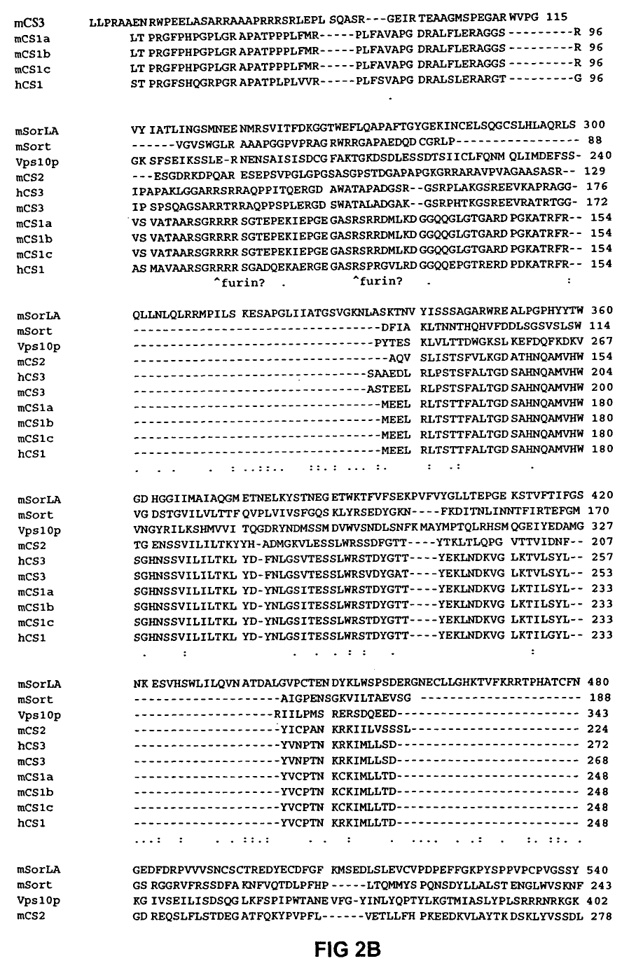

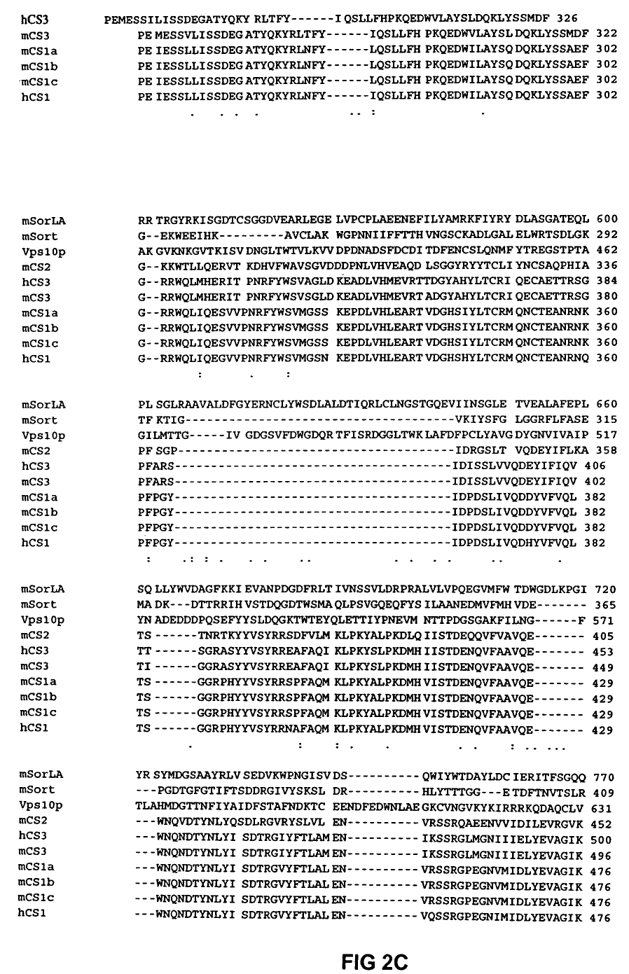

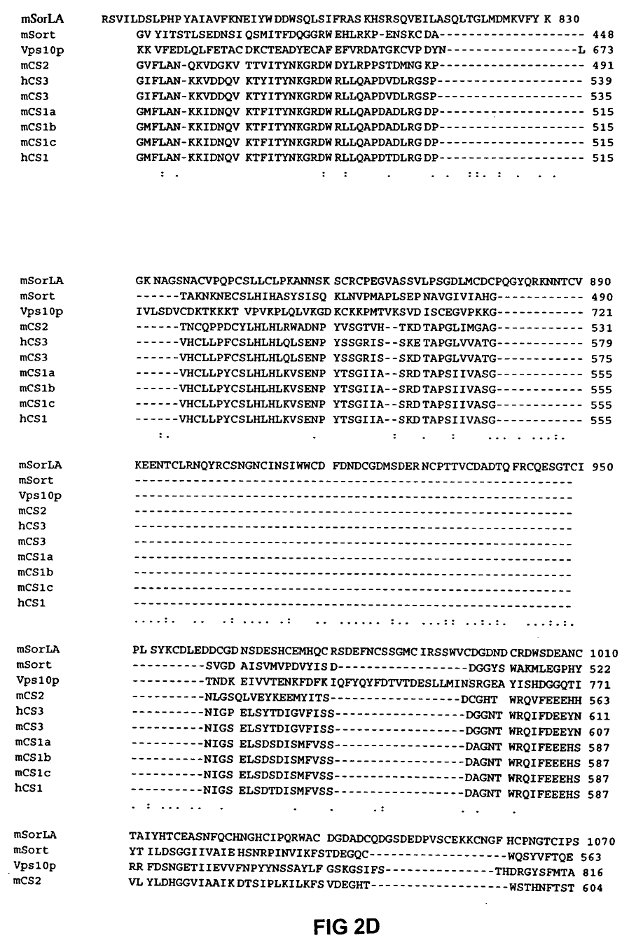

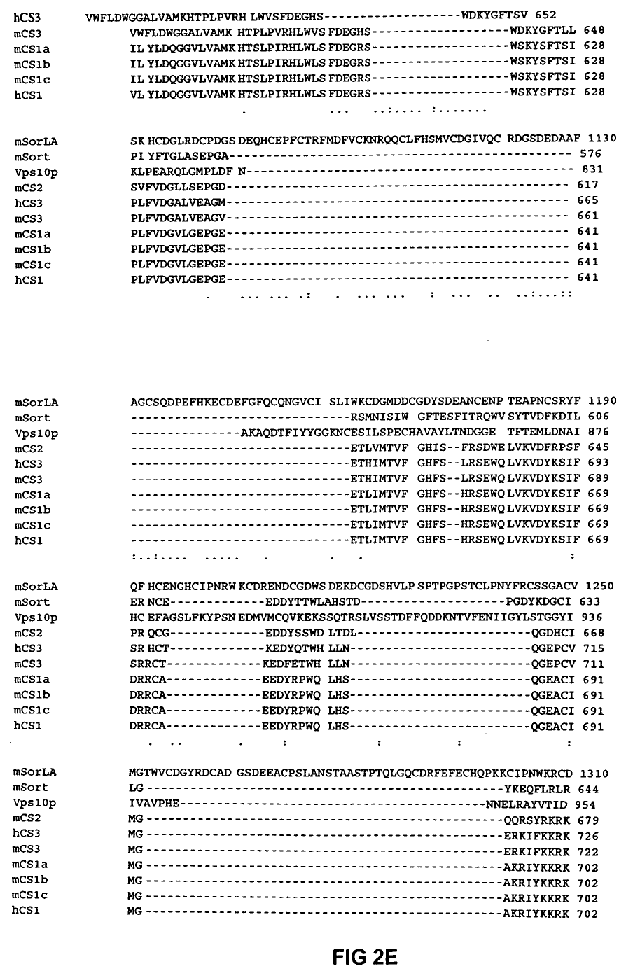

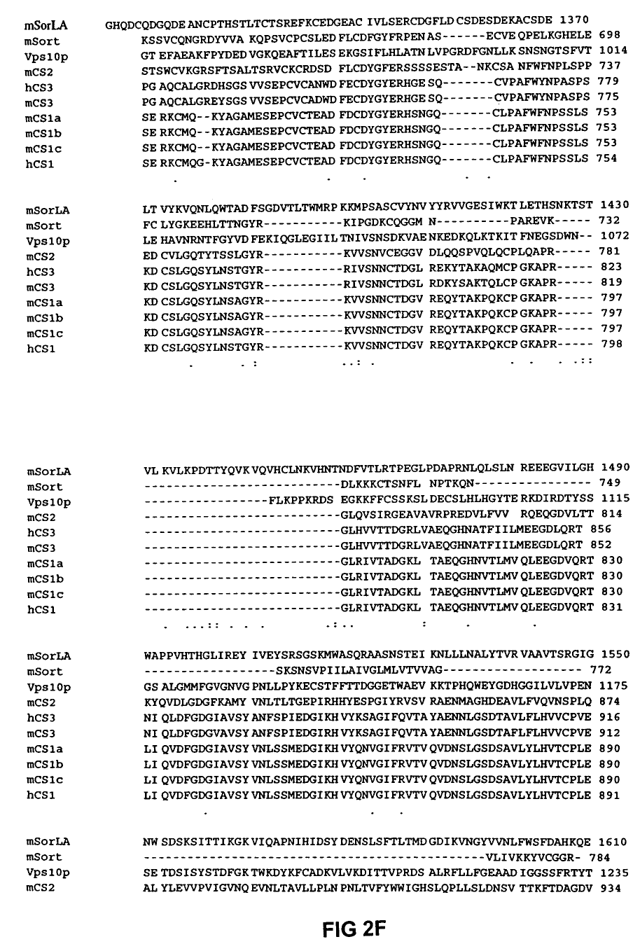

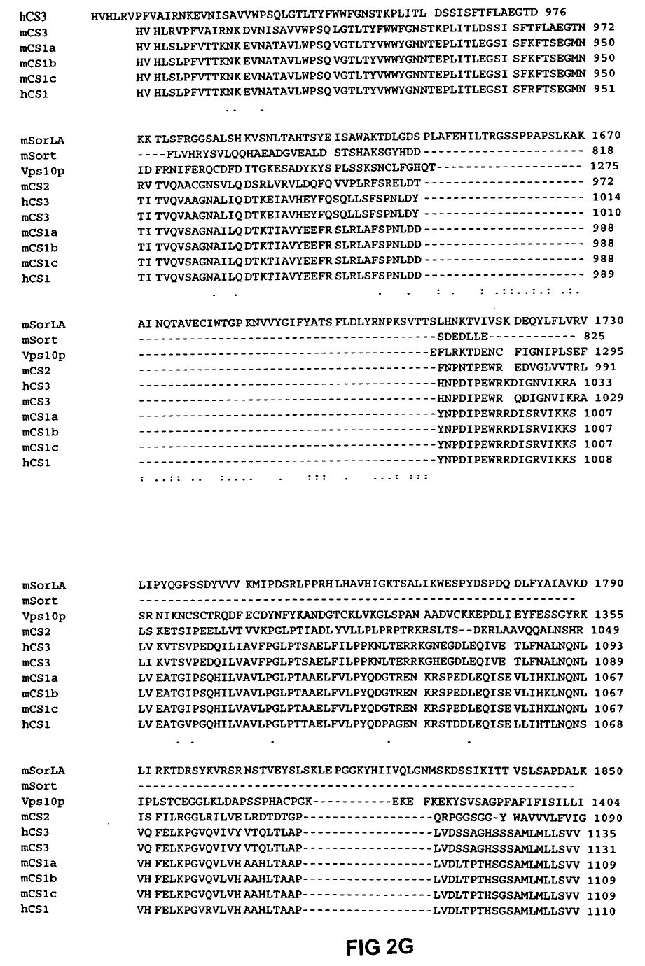

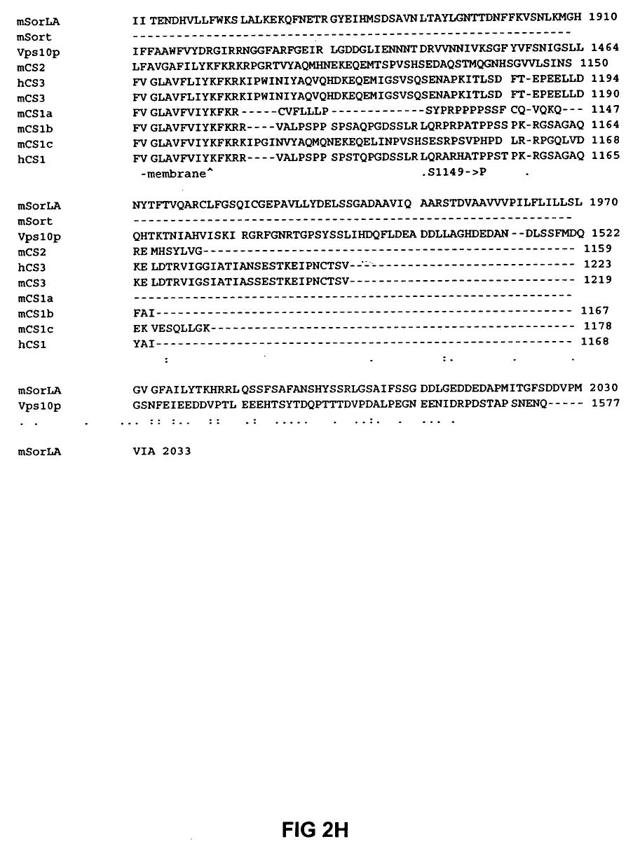

The genomic and cDNA sequences of human SorCS1 is known. The human SorCS1 cDNA sequence (GenBank Accession No. NM_{—}052918) and SorCS1 amino acid sequence (GenBank Accession No. NP_{—}443150) are incorporated herein as SEQ ID NO.1-2 respectively. Also, shown in the sequence attachment hereto are the amino acid sequences of mouse (SorCS1a, SorCS1b, SorCS1c) and human (SorCS1a, SorCS1b, and SorCS1c). Also shown in FIG. 2 is an amino acid sequence alignment between mouse SorCS1b (mSorCS1b) and human SorCs1 (hSorcs1). Note that the sequences are highly homologous, in fact have a sequence identity of 93%. It is this degree of identity that provides the rational for the prediction that the genetic evidence from the congenic mouse model presented here does, in fact, predict the same genetic phenomenon in humans.

From a diagnostic perspective, individual human beings can be examined for the allele of their SorCS1 gene as a step in determining whether they are susceptible to developing type 2 diabetes. For example, the SorCS1 cDNA sequence of an individual can be determined and the deducted amino acid sequence can be compared to SEQ ID NO:2. If a mutation at the amino acid level is detected, especially if the mutation is one other than a conservative substitution, the individual can be identified as susceptible to developing type 2 diabetes.

We have also discovered a similar allelic difference associated with susceptibility to diabetes, in a highly related gene. We have detected a C-to-A mutation in the SorCS3 gene which is found in a Bedouin Arab family in which type 2 diabetes has a high occurrence rate. The mutation results in a Serine to Arginine mutation at amino acid 790 of the human SorCS3 amino acid sequence (SEQ ID NO:4). While it is thus known that this mutation can permit the development of type 2 diabetes, there are certainly other mutations of these genes which cause the same susceptibility. The discovery of this mutation lends support to the concept that both of the related genes SorCS1 and SorCS3 can be the source of genetic susceptibility to type 2 diabetes.

Susceptibility may also be determined by measuring the mRNA or protein level of SorCS1 or SorCS3. A lack of expression of the proper form of SorCS1, SorCS3, or both, at either the mRNA level or the protein level indicates susceptibility to developing type 2 diabetes. The expression level can be compared to the normal range of level of expression and a expression level than the normal range indicates susceptibility to developing type 2 diabetes. The normal range of level of expression can be established by measuring the expression level in a suitable number of type 2 diabetes-free individuals. Given that the cDNA and amino acid sequences of both SorCS1 and SorCS3 are known, one of ordinary skill can readily design probes and primers and generate antibodies to practice the method described above.

Diagnostic analysis of the SorCS1 or SorCS3 gene may also be valuable in the field of pharmacogenomics. Some therapeutic agents are only effective in patients having a selected variant of a certain gene. In this embodiment, a subject in need of treatment provides a DNA sample from which the DNA sequences of SorCS1 and SorCS3 are determined. The outcome determines which therapeutic agent is administered to the patient.

From the perspective of prevention and treatment of type 2 diabetes, natural or non-natural ligands of SorCS1 or SorCS3 that modulate (i.e. stimulate or antagonize) the activity of the proteins are potential prevention and therapeutic agents. SorCS1 and SorCS3 are cell surface receptors which presumably trigger a cellular process. If this process can be stimulated artificially, the effect of the disease might be ameliorated. For example, when an individual does not produce enough natural ligands for SorCS1 or SorCS3, the natural ligand or an artificial ligand can be administered into the individual to bind to and increase the function of the receptor. In addition, if the SorCS1 or SorCS3 pathway does not function, increasing the activity by administering a ligand may help compensate for the lost function. Neurotensin, which binds to sortilin on the VPS10 domain, is expected to bind to SorCS1 and potentially can be used as a preventive or therapeutic agent for type 2 diabetes.

Other natural or non-natural ligands of SorCS1 or SorCS3 can be identified. Given that the cDNA and amino acid sequences of SorCS1 and 3 are known, one of ordinary skill can readily screen for agents that interact with SorCS1 or SorCS3. For example, one can use a cell culture system in which cells express SorCS1 or SorCS3. These cells can be exposed to a test agent and the presence and absence of an agent/SorCS1 complex is determined. It is well within the capability of one of ordinary skill in the art to make such a determination. An in vitro system in which a SorCS1 or SorCS3 protein can be exposed to a test agent directly can also be used to screen for ligands of SorCS1 or SorCS3. In the screening method described here, not only the human SorCS1 but also mouse SorCS1, or genes from other mammalian species can be used. Fragments of these proteins that include the VPS10 domain or other domains can also be used. Mouse SorCS1 mRNA and amino acid sequences are available at GenBank Accession No. AF195056.

All types of assays for identifying ligands and modulators of SorCS1 or SorCS3 are contemplated by the inventors. Such assays include, but are not limited to, assays which measure SorCS1 or SorCS3 biological activity, assays which measure expression of SorCS1 or SorCS3 (preferably employing the promoter gene sequence of these proteins linked to a reporter gene) or “in silico” assays which use computational models of the protein to predict compounds which will modulate the protein biological activity or expression. The assays are designed to identify ligands and modulators which are potential therapeutic agents, or analogs thereof, which have utility in the treatment of type II diabetes and related diseases.

mRNA or protein expression assays are also useful for identifying compounds which can modulate (i.e. up regulate or down regulate) expression of the gene, including compounds which modulate the activity of transcription regulators of SorCS1 or SorCS3. Such expression assays typically include an expression construct comprising the promoter region (5′UTR and associated genomic sequence) of the gene linked to a reporter gene. Potential therapeutic agents, or analogs thereof, are identified by their ability to modulate expression of the gene in question. Those skilled in the art are capable of identifying transcription factors which are responsible for regulating transcription of the gene in question.

Ligands and modulators identified for use as therapeutic (or prophylactic) agents can be of any composition. They are preferably orally available small molecule compounds. In an alternative embodiment, such compositions are selected from among small molecules, antisense molecules, siRNA, therapeutic antibodies and the like. In some embodiments a gene therapy vehicle (plasmid, viral or non-viral (lipid based) vector) may be used to deliver a copy of the SorCS1 gene to a cell for therapeutic expression of the respective proteins. Therapeutic compounds may be delivered orally, intravenously, by inhalation, and/or by any other of the means well known to those in the art.

The invention also includes a wide variety of tools for use in research which employ SorCS1 or SorCS3, such as but not limited to purified genes or proteins, recombinant cells containing additional copies of the gene(s), antibodies to the proteins (humanized, therapeutic or otherwise) and transgenic animals, such as mice created to have non-functional forms of the gene (knock-out or knock-down) or recombinant mice having additional copies of the gene(s).

EXAMPLE

As described in Stoehr, JP et al., Diabetes 49: 1946-1954 (2000), which is herein incorporated by reference in its entirety, when the t2dm2 locus on chromosome 19 of the C57BL/6 (B6) mouse strain was introduced into the BTBR mouse strain background to generate congenic mice, the non-diabetic BTBR mice became more severely diabetic. The inventors here generated a panel of interval specific congenic strains (ISC strains) for the t2dm2 locus on chromosome 19 of the B6 mouse in the BTBR background. The diabetic phenotype of the ISC strains were determined by measuring the fasting glucose levels. By comparing the overlapping t2dm2 locus fragments contained in different ISC strains and their phenotype, the genomic region that contains the type 2 diabetes susceptibility gene(s) was narrowed down to a 7 Mb fragment. Through searching the sequence information available from Celera and Public Genome Consortium, one gene of the size of about 0.5 Mb called SorCS3 was identified in the region. This gene is present in both mouse and the syntenic region of the human genome (chromosome 10). The full-length mRNA for this gene has been detected in both humans and mice. Close by SorCS3, in both human and mouse, a gene called SorCS1 that belongs to the same sortilin family as the SorCS3 gene was found. Both SorCS3 and SorCS1 were suspects for possible type 2 diabetes susceptibility genes found in this region.

Triglyceride levels in congenic mice of B6/7 Mb in the BTBR background were measured. The homozygotes, which were diabetic, were found to have a higher triglyceride level than the heterozygotes, which were non-diabetic.

Subsequent sequencing of the alleles of the SorCS1 and SorCS3 genes in the two strains led to the identification of SorCS1 as the responsible genetic differentiation. This conclusion was reached because the alleles of the SorCS3 gene in the two mouse strains was identical, where, but contrast, the alleles of the SorCS1 genes differed from each other by three nucleotides, as identified above in Table 1.

It appears that the activity of the SorCS1 protein may determine islet mass. Alternatively, the SorCS1 protein may affect insulin secretion in pancreatic beta cells or insulin degradation in the kidney or liver. Either of these will affect plasma insulin levels, which are altered in the congenic mice.

### REFERENCES

1. Kahn B B: Type 2 diabetes: when insulin secretion fails to compensate for insulin resistance. Cell 92:593-596, 1998

2. Taylor S I: Deconstructing type 2 diabetes. Cell 97:9-12, 1999

3. Polonsky K S, Sturis J, Bell G I: Non-insulin-dependent diabetes mellitus: a genetically programmed failure of the beta cell to compensate for insulin resistance. N Engl J Med 334:777-783, 1996

4. Polonsky K S: The beta-cell in diabetes: from molecular genetics to clinical research. Diabetes 44:705-717, 1995

5. Kahn C R, Vicent D, Doria A: Genetics of non-insulin-dependent (type-II) diabetes mellitus. Annu Rev Med 47:509-531, 1996

We claim:

1. A method of assessing whether a human subject is susceptible to type 2 diabetes comprising the step of determining the allele in the genome of that subject of the SorCS1 or SorCS3 gene.

2. A method of assessing whether a human subject is susceptible to type 2 diabetes comprising the step of analyzing the nucleic acid sequence of the subject in the SorCS1 or SorCS3 gene.

3. A method for determining whether a human being is a candidate for developing type 2 diabetes, the method comprising the steps of:
determining the sequence of the protein coding region of the SorCS1 or SorCS3 gene of the human being;
deducing the amino acid sequence encoded by the region sequenced; and
comparing the amino acid sequence to SEQ ID NO:2 or SEQ ID NO:4, respectively, wherein a difference observed indicates the human being as a candidate for developing type 2 diabetes.

4. A method for determining whether a human being is a candidate for developing type 2 diabetes, the method comprising the step of:
determining the mRNA or protein expression level of either SorCS1 or SorCS3 in the human being wherein the expression in comparison to normal range level of expression established by type 2 diabetes-free individuals indicates that the human being is a candidate for developing diabetes.

5. A method for identifying an agent that interacts with SORCS 1 protein, the method comprising the steps of:
exposing a SORCS 1 protein to a test agent; and
determining whether the test agent binds to the SORCS 1 protein.

6. The method of claim 5, wherein the SORCS 1 protein is from a human, a mouse or a rat.

7. A method for preventing or treating type 2 diabetes in a human being, the method comprising the step of administering neurotensin to the human being in an amount sufficient to prevent or treat type 2 diabetes.

8. A method for identifying a therapeutic agent, or analog thereof, which is useful for the treatment of type 2 diabetes and related diseases, the method comprising the steps of:
exposing a SORCS 1 protein to a test agent; and
determining whether the test agent modulates the biological activity of SORCS 1 protein.
